(19) United States
(12) Patent Application Publication
(10) Pub. No.: US 2007/0042412 A1
(43) Pub. Date: Feb. 22, 2007

(54) Detection of biologically active compounds

(76) Inventors: Dmitri Boris Papkovsky, Blarney (IE); Paul O'Sullivan, Carrigaline (IE); Martina Burke, Galway (IE); Tomas O'Riordan, Cork (IE)

Correspondence Address: JACOBSON HOLMAN PLLC, 400 SEVENTH STREET N.W., SUITE 600, WASHINGTON, DC, 20004, US

(21) Appl. No.: 11/506,209

(22) Filed: Aug. 18, 2006

Related U.S. Application Data
(63) Continuation of application No. PCT/IE05/00018, filed on Feb. 21, 2005.

(30) Foreign Application Priority Data
Feb. 19, 2004 (IE) IE2004/0105

(51) Int. Cl. C12Q 1/68; C07D 487/22; C07H 21/04

(52) U.S. Cl. 435/6; 536/25.32; 540/145

(57) ABSTRACT

A probe comprises a supramolecular structure having a chemical or biological recognition moiety; a phosphorescent reporter label; and an effector which interacts with the label so that the probe alters its phosphorescent characteristics on recognition of a target. The phosphorescent reporter label may have an emission lifetime in the order of 1 μs to 10 ms and may be selected from phosphorescent tetrapyrrolic compounds and their metallocomplexes.

### INTRODUCTION

The invention relates to the detection of biologically active compounds, particularly specific nucleic acid sequences such as DNA and RNA and other biomolecules such as polypeptides and enzymes.

### BACKGROUND

Detection and quantification of biologically active compounds is an important analytical task. The development of corresponding methods and reagents which allow simple, rapid, sensitive and cost-efficient detection of target biomolecules, such as specific DNA and RNA sequences or protein markers is of high practical need. Homogeneous (separation-free) bioaffinity assays using target-specific probes based on photoluminescent labels that alter their emission in the presence of the target provide efficient solutions to this task.

A number of schemes and measurement principles have been described, in particular for the detection of nucleic acids in solution without the need to separate or purify the target. Such assays, which are usually coupled with the process of amplification of target nucleic acid sequence using polymerase chain reaction (PCR) or alternative schemes, are often called “real-time PCR” schemes. They usually employ specially designed oligonucleotide probes labelled with a fluorescent dye or a pair of dyes, which alter their emission properties upon recognition and hybridization to the target nucleic acid sequence. Many such probes and assay formats employ the effects of close proximity quenching between pairs of labels/dyes which are incorporated in the structure of such probe(s). Recognition by the probe of the target sequence changes the effective distance between the labels, thus probe fluorescence and allows monitoring of target amplification during the PCR process and quantification of target concentration. In many cases, the main mechanism of proximity quenching in such probes is fluorescence resonance energy transfer (FRET) between the two labels.

Examples of such assays include the use of pairs of probes single-labelled at their 3′- or 5′-end, which hybridise to the target sequence adjacent to each other (EP0070685 A2). Alternatively, the two probes are complementary to each other and form a ‘dark’ complex, which is dissociated by the target (EP 0232967A2). In these schemes, recognition of target sequences by the probes and hybridization to them change, either increase or decrease the effective distance between the two labels attached to these probes, thus changing the efficiency of FRET and hence the signal of reporter dye (quenching or enhancement of fluorescence), which is monitored by a suitable detection system. The limitations of these schemes are relatively small signal changes upon target recognition, limited distance between two labels, complex assay procedure and limited flexibility with the probe design.

Other common formats of real-time PCR assays employ dual-labelled probes, for example TaqMan® (U.S. Pat. No. 5,210,015 and U.S. Pat. No. 5,538,848), “molecular beacons” (U.S. Pat. No. 5,925,517). In the TaqMan® format the probe is labelled at its 5′- and 3′-ends with the fluorescent dye and the quencher. The probe is designed to be relatively short to allow efficient FRET between the two dyes, so that the probe becomes weakly fluorescent. Being incorporated in the PCR amplification performed with a special enzyme Taq polymerase, the probe hybridizes to the target sequence generated in the PCR where it is cleaved by the enzyme which also has 5′-exonuclease activity. As a result, the fluorophore and the quencher are separated (released in solution). This causes an increase in fluorescence signal which is proportional to the amount of target sequence present in the sample and/or the number of amplification cycles. However, this scheme is limited to short probes (usually 16-30 bases). It produces moderate signal changes during amplification and requires probe cleavage which occurs only with certain polymerase enzymes.

The ‘molecular beacons’ format operates with longer probes, in which the two labels are also attached to the ends of a nucleic acid sequence. Such a probe is relatively long, it contains a sequence specific to target DNA and also short (4-7 nucleotides) self-complementary sequences on both ends (U.S. Pat. No. 5,925,517). In the absence of target the probe normally forms a hairpin confirmation with a characteristic stem region. This conformation ensures efficient FRET, as the two labels bound to 3′- and 5′-ends of the probe are brought in close proximity to each other. In the presence of target, the probe hybridizes to it with high affinity, opens the hairpin structure and linearises itself. This process separates the two dyes, reduces FRET and causes signal enhancement upon hybridization. Quenching can be eliminated by heating the probe above melting temperature of the stem region, thus opening the hairpin structure. A modification of this method, which also operates with dual-labelled hairpin probes is described in U.S. Pat. No. 6,150,097. Fluorescent reporter and quencher groups are attached to both ends of oligo, interacting with each other by means of a direct contact (non-FRET mechanism). This also causes efficient quenching of the probe in the absence of target and signal enhancement upon hybridisation. The limitations of such probes are the need for additional fragments (stem region), relatively complex design and structural requirements for such probes (e.g. melting points, composition) and competition between probe hybridization to the target sequence and to self.

Modifications of assay formats described above include the use of alternative amplification schemes such as strand displacement amplification. To enable the detection of RNA, PCR amplification is usually coupled with reverse transcription using an appropriate reverse transcriptase enzyme. Detection principles for such schemes and probe design remain rather similar to those described above.

The existing probes and formats of real-time PCR usually rely on conventional short-decay fluorescent labels and classical FRET pairs (i.e. donor and acceptor). There are limited possibilities in multiplexing of such assays, as the use of more than three fluorescent labels/probes in one assay tube is very difficult if not impossible, due to overlapping of fluorescence spectra and cross-interference.

Similar assay methodology and probe design are used for measurement of the activity or inhibition of some enzymes. In these cases, fluorescently labelled oligopeptide substrates and FRET schemes are usually employed. Such probes alter their fluorescence as a result of cleavage or chemical modification by the enzyme, which can be monitored in that way.

The invention is directed towards providing a range of new probes and corresponding assay methods which will at least assist in extending the range of applications of homogeneous bioaffinity assays and in overcoming some of their existing problems and limitations.

### STATEMENTS OF INVENTION

According to the invention there is provided a probe comprising a supramolecular structure having:
a chemical or biological recognition moiety;
a phosphorescent reporter label; and
an effector,
in which probe the label interacts with the effector so that the probe alters its phosphorescent characteristics on recognition of a target.

In one embodiment of the invention the phosphorescent reporter label has an emission lifetime in the order of 1 μs to 10 ms. Preferably an emission lifetime in the order of 10 μs to 1000 μs.

In one embodiment of the invention the phosphorescent reporter label is selected from a group of phosphorescent tetrapyrrolic compounds and their metallocomplexes. The phosphorescent reporter label may selected from any one or more of phosphorescent metallocomplexes of porphyrins, chlorins, porphyrin-ketones and related structures.

The phosphorescent label may be selected from any one or more of platinum(II)-porphyrin, platinum(II)-coproporphyrin, palladium(II)-porphyrin and palladium(II)-coproporphyrin.

In one embodiment of the invention the phosphorescent label is in the form of a monofunctional labelling reagent.

In one embodiment of the invention the effector is selected from any one or more of dabcyl, QSY-7™, ‘black hole quenchers’™, rhodamine green, FITC, Cy5, and analogs thereof.

In one embodiment of the invention the effector comprises a small-size chemical structure. Preferably a chemical structure less than 300 Daltons in size. In this case the effector may be selected from any one or more of dinitrophenol, a nitrophenol moiety and derivatives thereof.

In one embodiment of the invention the effector is a modified nucleotide base.

In one embodiment of the invention the phosphorescent reporter label and the effector are both provided by the same chemical structure. Preferably the reporter label and the effector both comprise a phosphorescent metalloporphyrin label.

In one embodiment of the invention the recognition moiety is a common biomolecular structure or a biopolymer.

The invention also provides a probe as hereinbefore described further comprising a spacer(s) linking the recognition moiety, the reporter label and the effector. Preferably the spacer(s) is 2 to 18 atoms in length.

In one embodiment of the invention the reporter label is attached to one of the termini of a biopolymer. The biopolymer functions as the recognition moiety

In one embodiment of the invention the recognition moiety comprises a biopolymer with the reporter label attached to one of its termini and the effector attached to the other termini.

In a further embodiment of the invention the recognition moiety comprises a biopolymer with the effector attached to one of its termini and the reporter label attached internally.

In a preferred embodiment of the invention the probe is quenched in its free form in solution.

In another embodiment of the invention the chemical or biological recognition moiety comprises a single-stranded oligonucleotide sequence. In this case the probe produces a phosphorescent signal response upon recognition of a complementary target, hybridisation and formation of a double-stranded structure with the target.

Preferably the reporter label and the effector are attached to the 5′- and 3′-ends respectively of the specific nucleic acid sequence.

In one embodiment of the invention the reporter label is attached to the 5′-end of the probe and the effector is incorporated internally or attached to one of the bases inside the probe sequence.

Preferably the probe is 15 to 100 bases long, most preferably 20 to 50 bases long.

In one embodiment of the invention the probe has the ability to hybridise to a target and act as a primer in the process of elongation of the polynucleotide chain by polymerase enzymes using the complement as a template.

In one embodiment of die invention the reporter label is platinum(II)-porphyrin and the internal effector is a modified nucleotide base.

In a further embodiment of the invention the chemical or biological recognition moiety comprises an oligopeptide sequence. In this case quenching of the reporter label is affected by probe cleavage associated with the recognition process. Preferably the probe is cleaved or modified by a specific enzyme.

In one embodiment of the invention the chemical or biological recognition moiety comprises a structure acting as an intrinsic quencher for the reporter label. The intrinsic quencher for the phosphorescent metalloporphyrin label may be a tyrosine residue within an oligopeptide sequence.

In another embodiment of the invention the intrinsic quencher for the phosphorescent porphyrin label is a histidine residue within an oligopeptide sequence.

In one embodiment of the invention the chemical or biological recognition moiety comprises a polysaccharide or a peptide nucleic acid.

One aspect of the invention provides a probe comprising a chemical or biological recognition moiety; a long decay photoluminescent reporter moiety; and a quencher moiety, wherein the probe alters its photoluminescent signal on recognition of a target molecule. Preferably the reporter moiety is a long-decay phosphorescent label which is quenched by the quencher moiety mostly by a static mechanism(s) but not by resonance energy transfer.

The invention also provides a method for the detection of a chemical or biological species comprising the steps of:
providing a probe as claimed in any preceding claim;
exposing the probe to a sample containing a target species;
measuring the phosphorescent response of the probe on recognition of the target; and
qualifying and quantifying the target based on the measured phosphorescent signal.

In one embodiment of the invention the method comprises preparing a solution comprising the probe and mixing the probe solution with a sample solution containing a target.

In one embodiment of the invention the target comprises a nucleotide sequence.

In another embodiment of the invention the method comprises the recognition of a target sequence by the probe, amplification using a set of primers specific to a particular region of the target nucleotide sequence and a polymerase chain reaction.

In one embodiment of the invention the probe also acts as a primer.

In one embodiment of the invention the probe is used to distinguish between complementary and non-complementary target nucleotide sequences.

In another embodiment of the invention the probe is used to distinguish between a perfect complement and a single-point mismatch or polymorphism.

Preferably the target amplification and detection are carried out in a closed tube format.

The invention further provides use of a probe of the invention in hybridisation, binding and enzymatic assays, especially homogenous assays.

In one embodiment of the invention the assay is based on the use of close proximity quenching of a long-decay phosphorescent label.

The term supramolecular structure is taken to mean a structure with at least two distinct chemical moieties/fragments linked by means of chemical bonds to each other or to a common backbone. The term supramolecular includes the term tri-functional.

A tri-functional probe is taken to include probes which are dual-labelled or single-labelled. In the case of single-labelled probes the effector may be internal. Dual-labelled probes may comprise two identical or similar labels.

### BRIEF DESCRIPTION OF THE DRAWINGS

The invention will be more clearly understood from the following descriptions thereof given by way of example only, with reference to the accompanying drawings, in which:

FIG. 1 is a schematic representation of a probe according to the invention. (R—phosphorescent reporter moiety, Q—quencher moiety linked to the recognition moiety. (linkers are shown as bars). Signal change is produced upon probe chemical modification or cleavage (e.g. by enzyme), or conformational change (e.g. due to binding or hybridisation to the target).

FIG. 2 are graphs for comparative purposes showing the characteristic quenching behaviour of the platinum(II)-coproporphyrin (top) and palladium(II)-coproporphyrin (bottom) labels (also referred to as PtCP and PdCP respectively or MeCP) attached to an 18-mer oligonucleotide upon hybridization with complementary oligos labelled with different quenchers (indicated on each graph) located at different distances from 0 to 18 base pairs away from the porphyrin label;

FIG. 3 is a graph showing the absorption spectra of tri-functional oligonucleotide probes bearing the phosphorescent PtCP label and QSY-7™ (bold line), dabcyl (solid line) and Cy5™ (dashed line).

FIG. 4 is a graph showing the degree of quenching of the PtCP label by different quenchers in the tri-functional 23-mer oligonucleotide probe in single-stranded conformation;

FIG. 5 is a graph showing phosphorescence enhancement of a tri-functional single-stranded 23-mer oligonucleotide probe upon its hybridisation to target complementary sequence and formation of double-stranded structure in solution. Conditions: 50° C., 10 mM tris buffer containing 50 mM KCl, 1.5 mM MgCl₂, 100 mM Na₂SO₃, pH 7.8. A—The point of addition of probe to buffer, B—The point of addition of 2-fold molar excess of complementary sequence to test sample;

FIG. 6 is a bar chart showing the dependence between the length of tri-functional phosphorescent oligonucleotide probes and phosphorescence enhancement upon hybridisation with complementary oligonucleotides or digestion by non-specific nuclease enzyme;

FIG. 7 is a bar chart showing the enhancement of the phosphorescent signal upon hybridization of the tri-functional oligonucleotide probe to its target at different temperatures and the effect of single base mismatch in target sequence;

FIG. 8 is (a) Agarose gel electrophoresis of TB1-PtCP-labelled oligonucleotide probe incorporated into PCR amplification. Lanes 1-5 contain PCR product amplified in the presence of PtCP-QSY-7-labelled 18mer, 21mer, 23mer, 25mer and 30mer respectively. Lanes 6 and 7 are negative and positive controls, respectively and lane 8 is a 100 bp molecular weight marker, (b) Corresponding measurement of PCR samples on the Victor² plate reader. I/I₀ values are determined by dividing the signal from the positive sample by that of a negative PCR control containing the same concentration of probe but no template DNA was added;

FIG. 9 is a graph showing the change in phosphorescence of the tri-functional oligonucleotide probe bearing reporter PtCP label during the PCR;

FIG. 10 is an absorption spectrum of the peptide Ac-CDEVDAPK-NH2 labelled with PtCP and dabcyl;

FIG. 11 is a bar chart showing phosphorescence enhancement of the peptide probe of FIG. 10 due to its cleavage by caspase-3 enzyme induced in apoptotic cells; and

FIG. 12 is a graph showing the sensitive and selective detection of the probe of the invention by time-resolved fluorescence on a Victor V plate reader (excitation/emission filters—340/642 nm, delay time—30 us, gate time—100 us).

### DETAILED DESCRIPTION

The invention provides a range of probes based on phosphorescent labels and corresponding assay formats, which allow for the detection of biological molecules such as specific nucleic acid sequences, proteins and other targets in solution, without the need for separation of assay components. These probes and assay formats have been developed and optimised particularly for use in separation-free hybridisation assays coupled with nucleic acid amplification (so-called real-time PCR formats) and for measurement of the activity and inhibition of certain enzymes and ligand-receptor interactions in homogeneous formats.

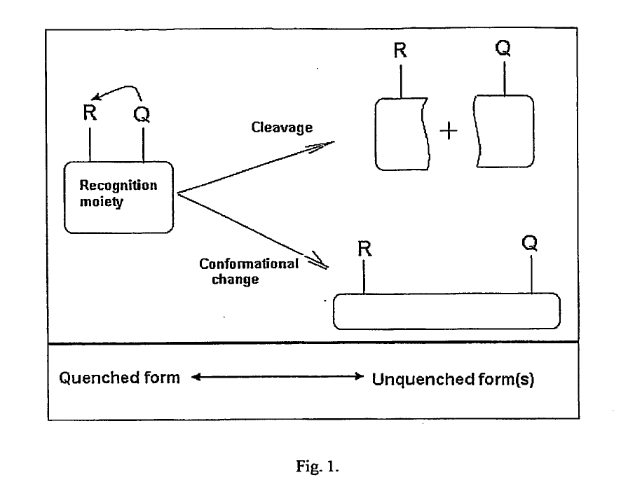

The probe of the invention comprises a supramolecular structure comprising the following units: a moiety which can participate in a process of specific recognition of its chemical or biological target, so that said recognition alters the conformation or chemical composition of the probe as a whole; a phosphorescent reporter label with relatively long lifetime; an effector moiety which has an enhanced quenching effect on the reporter label in certain conformation(s) or modifications of the probe and a reduced quenching effect in other conformation(s) or modifications. The probes may also comprise ancillary units such as linkers and spacers which connect these moieties together and provide them with optimal spatial orientation, dynamics and functional properties under assay conditions. As a result of such organization, the probe produces a distinct phosphorescent signal or signal change upon recognition of its target, which can be used for the identification and quantification of the target in a sample. Usually, recognition involves binding of the probe to its target, chemical modification or cleavage of the probe, which normally take place in solution and which affects the degree of interaction between the phosphorescent reporter and the effector/quencher moieties. The effector/quenching moiety may be an extrinsic chemical moiety or an intrinsic chemical moiety within the recognition structure having a well-defined location (usually some distance away from the reporter label) and occurs at relatively low abundance. The general design and mode of action of a probe of the invention is presented schematically in FIG. 1.

The probes of the invention are distinct with respect to their composition, photophysical properties and quenching behaviour to probes described before. When used in bioanalytical applications and particularly in homogeneous bioaffinity assays, the probes display a number of advantageous features in comparison with established probes and assays. The probes also allow a number of new assay formats and applications which were not possible or were inefficient using conventional fluorescent labels and probes. The probes also allow multiplexing with some existing probes and simultaneous detection of several targets in one sample.

One of the important and characteristic features of the probes of the invention is the characteristic photophysics of their reporter labels and long emission lifetime, which exceeds the lifetime range of conventional fluorescent probes (typically 1-10 ns) by several orders of magnitude. Due to these features, the mechanisms of close proximity quenching, molecular organisation and dynamics of such probes are quite different from those of probes employing other photoluminescent labels such as conventional fluorophores. These features of the probes have a large impact on their general design, photophysical behaviour and the ability to modulate their emission upon target recognition.

One of the key features of the probes of the invention is that the general photophysics of their emission is very different from those based on conventional fluorescent labels, which is due to the differences in their excited state pathways and transitions. Conventional fluorophores are usually excited into their first excited singlet state and then emit back from this state (i.e. So→S₁ and S₁→So transitions, respectively). Conversely, the phosphorescent labels emit from their excited triplet state (T₁→Sₒ transition), which is produced in the course of several intermediate transitions. Furthermore, phosphorescent metalloporphyrins are excitable with visible light into S₁ (the Q-bands) or with UV light into S₂ (the Soret band). Following the absorption of a photon of light, the phosphorescent molecule undergoes internal conversion, intersystem crossing and relaxation processes which involve different electronic and energy states and which eventually produce the long-lived excited triplet state from which emits phosphorescence (S₂→S₁→T₁→S₀). Such complex photophysics of phosphorescence in general and metalloporphyrins in particular has a marked effect on the probe/label photophysical properties. These effects become more pronounced for complex macromolecular structures in which the phosphorescent label may be involved in interactions with other chemical structures and in processes such as quenching, resonance energy transfer, complex formation. Additional spin factors and restrictions (phosphorescence itself and some intermolecular processes involving triplet states are forbidden by spin) as well as probe microenvironment and conformational dynamics during the time of excited state also largely contribute to this. As a result, the phosphorescent labels display characteristic behaviour in the schemes used in homogeneous assays, in particular in assays that use close proximity quenching formats.

The long-lived excited triplet states of the labels used in these probes are also prone to interactions with different chemical structures which may be present in the probe and in the sample. For example, the phosphorescent labels of the invention were shown to be effectively quenched by various chemical structures. Such extrinsic quenchers can be incorporated in the macromolecular structures together with the reporter label to produce the probes of the invention. At the same time, the labels and probes are not quenched or minimally quenched by chemical structures which may be present in the recognition moiety of the probe (e.g. nucleotide bases, amino acids), nor by sample components (e.g. solvents, buffer components, proteins, mononucleotides, polymerase enzymes, natural metabolites and other additives) commonly present or used in bioaffinity assays including real-time PCR schemes or enzymatic assays. As a result, the degree of quenching of the reporter label in such a probe depends mainly on the nature of the reporter label and the quencher, probe molecular organisation and dynamics, and the recognition process which involves the target. Labels with very long emission lifetimes (above 10 ms) are not very suitable, as they may be quenched by undesirable species and processes and are also less convenient to measure.

The preferred probes of the invention are those having a reporter moiety comprising phosphorescent labels having emission lifetimes in the order of 1 μs-10 ms. In particular, platinum(II)- and palladium(II)-porphyrin labels, which are known to have strong room temperature phosphorescence in aqueous solutions and have lifetimes of about 100 μs and 1000 μs respectively, were shown to be among the most efficient reporter labels for the probes and assays of the invention. Other phosphorescent labels including structures related to metalloporphyrins such as metallocomplexes of chlorins, porphyrin-ketones, other tetrapyrrols as well as some other phosphorescent dyes having appropriate photophysical properties and lifetimes in the specified range may also be used.

The range of structures with strong quenching effect on the phosphorescent reporter moiety of the invention is relatively broad and include structures which do not normally act as efficient quenchers of conventional fluorescent labels. Among these quenchers, the most useful are small-size quenchers which minimally interfere with the biological recognition function of die probe. A number of common quenchers currently used with conventional fluorescent dyes, such as dabcyl, QSY™ and ‘black hole quenchers’™ may also be used.

In addition, self-quenching of the phosphorescent labels of the invention may be exploited to design the probes of the invention and corresponding homogeneous assays. Self-quenching of the phosphorescent dyes such as metalloporphyrins in solutions is known to be considerable, but it is concentration dependent and vanishes at submicromolar concentrations of the dye. We have shown that self-quenching of metalloporphyrin labels becomes greatly enhanced in the supramolecular structures (probes) of the invention, which contain two of these labels in close proximity to each other. In such probes, self-quenching becomes independent of the probe concentration and it remains strong within a broad concentration range down to nanomolar concentrations and below. Self-quenching of such probes is also affected by recognition processes (binding, cleavage) which alter probe conformation or structure. The self-quenching of the phosphorescent label in the probes of the invention is different than for the free dye in solution. The approach based on self-quenching of phosphorescence requires just one type of label acting both as the reporter and the quencher to be used in the probe. This allows the design of more simple probes (a second chemical structure used as a quencher becomes redundant), also the increased specific phosphorescent signal from the probe (from two labels) upon target recognition.

To date there has been limited research on the use of long-decay photoluminescent labels in biological applications, particularly for use in hybridisation assays, homogeneous assays and proximity quenching schemes. Fluorescent lanthanide chelates Nurmi J, et al.—Anal Chem. 2002, 74(14):3525-32), ruthenium complexes (Hurley D. J., Tor Y.,—J. Am. Chem. Soc., 2002, 124(44): 13231-41) and a few other dyes have been studied to some extent. However, these labels have quite different photophysics compared to the ones used in the invention. The long-lived emission of the fluorescent lanthanide chelates occurs from the central metal ion, which is surrounded by aromatic ligands serving as light harvesting antennas helping to absorb excitation light energy and passing it to the metal ion. This light is emitting from the moiety (inner electronic shells of the metal ion) which is effectively shielded from interaction with other chemical species including quenchers. For the long-decay fluorescent complexes of ruthenium and osmium (e.g. ruthenium polypyridines), emission arises from metal to ligand charge transfer absorption which then leads to the emission from the organic ligand.

In metalloporphyrin labels, emission occurs from the aromatic tetrapyrrolic macrocycle, while the central metal ion only alters intramolecular energetics and balance of different deactivation pathways. Phosphorescence from the porphyrin ring becomes a dominating pathway for Pt(II)- and Pd(II)-porphyrins, while fluorescence and other deactivation pathways become unfavourable. The emitting moiety of these labels is rather large and it is exposed to various intra- and inter-molecular interactions and processes such as collision, complex formation, quenching.

Phosphorescent metalloporphyrin labels have been described for use in hybridisation assays and DNA detection systems (O'Sullivan P. J., et al.—Nucleic Acid Res., 2002, E1-7). Such probes are however based on single labelled or bi-functional probes.

In the present invention we have found that certain dual-labelled or tri-functional probes bearing a phosphorescent reporter label, such as metalloporphyrin, in their structure behave very differently when used in separation free hybridisation assays. They display characteristic features, which allow the development of a range of new probes and assay formats, which were either not possible or inefficient to achieve using the established fluorescent labels and probes commonly used in such detection formats.

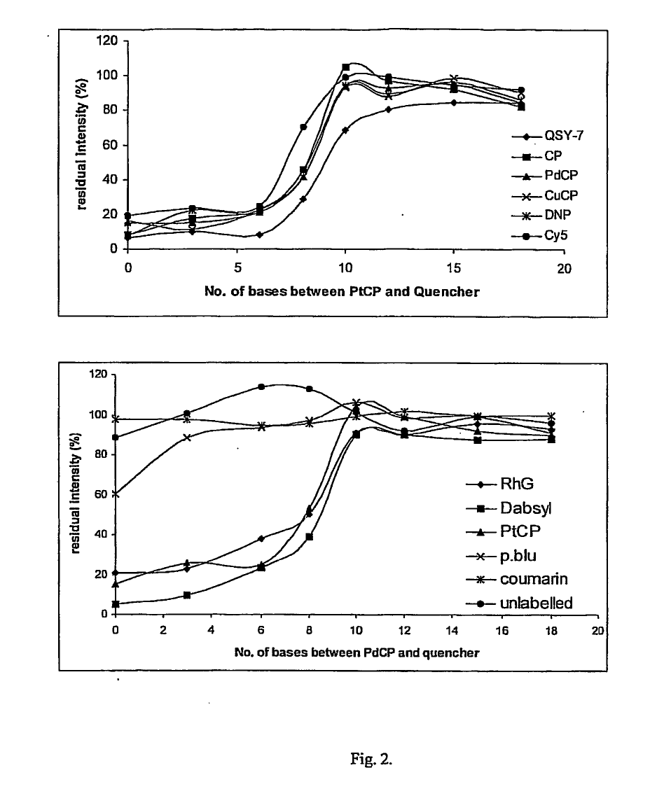

FIG. 2 shows the quenching of bi-functional oligonucleotides labelled with phosphorescent Pt- or Pd-coproporphyrin (MeCP) by complementary oligonucleotides labelled with the quencher upon their hybridisation and formation of double-stranded structure (see Example B). We have shown that in such systems the phosphorescent porphyrin labels are inefficient as resonance energy transfer (RET) donors. Several fluorescent dyes were tested as potential acceptors for Pt- and Pd-coproporphyrin labels, but no significant enhancement of acceptor emission was observed. On the other hand, a variety of different chemical structures were found to quench the phosphorescence of porphyrin labels when in close proximity. Quenching was found to be effective when the MeCP and quencher moiety were separated by distance up to 8 nucleotide bases, and was reduced when separation distance exceeded 10 bases. The absence of correlation between spectral overlap integrals and quenching efficiency, steep distance dependence and much smaller changes in emission lifetime of the donor suggest that quenching mechanisms are rather complex (mixed) in comparison to classical RET.

The tri-functional oligonucleotide probes of the invention, comprising two labels attached to their ends, wherein one of the labels is a phosphorescent metalloporphyrin label, were found to be quenched very efficiently in single-stranded conformation. For example, strong (3-30-fold) quenching was observed for 18-80-mer oligonucleotides labelled with Pt-coproporphyrin and QSY7™ at their 3′- and 5′-termini, respectively. Hybridisation of such ‘linear’ probes (i.e. without any stem region) to complementary sequences and formation of double-stranded structures, were found to drastically reduce quenching. Upon the addition of complementary target to a solution of ‘dark’ dual-labelled linear probe, large enhancement of the phosphorescence was observed, which correlated to the amount of target added. Non-specific sequences did not cause any significant signal changes of the probe. The exact structures, photophysical and quenching properties of such probes are described in more detail in the Examples.

Distance dependence of quenching for the tri-functional linear probes of the invention is also quite uncommon. The dependence of quenching on the probe length has a bell shape, with maximal quenching achieved at certain lengths of the probe, as seen in Example 2 (23-25 mer oligonucleotide). Quenching still remains fairly strong at much longer probe lengths. Conversely, in systems employing short-decay fluorescent labels and RET mechanism, distance dependence of quenching usually obeys function (1/R⁶) and vanishes very quickly with distance.

The relatively long-distance quenching effects with the phosphorescent labels may be associated with active conformational dynamics of the probe during the time when the phosphorescent label is in excited state. Quenching data (not shown) also suggests that stacking interactions and static quenching between the phosphorescent label and the quencher are playing a considerable role in the signal modulation upon target recognition. Long emission lifetimes in the micro- to millisecond range allow the macromolecular probe to pass through numerous conformations, some of which result in quenching of the reporter label. These effects are usually not observed or are less considerable for probes based on conventional (short-decay) fluorescent dyes.

Intramolecular photophysics and conformational dynamics of fluorescent probes has a much lower impact on quenching than phosphorescent probes. The short-lived excited states of fluorescent probes and simpler photophysics of their emission, limit their inter- and intra-molecular dynamics and the possibilities of quenching interactions involving such supramolecular structures. Fluorescence polarisation measurements with labelled proteins, nucleic acids and low molecular weight compounds also indicate that conformational dynamics for conventional fluorescent labels and their motion in solutions during the lifetime of their excited states is limited. As a result, to enhance the efficiency of quenching in dual-labelled oligonucleotide probes comprising fluorescent labels special modifications are used. For example, in ‘molecular beacon’ probes an additional ‘stem region’ is added to the probe at both ends to create a hairpin structure, which brings the two labels close to each other thus allowing effective FRET or physical contact between them.

In contrast to the ‘molecular beacon’ probes, hybridisation probes of the invention based on phosphorescent porphyrin labels do not require a stem region, as the quenching is efficient regardless. As a result, the use of phosphorescent porphyrin labels in the probes of the invention provide simpler ‘linear probes’ comprising two labels attached to die recognition structure.

TaqMan™ probes employing linear probe structures, fluorescent labels and RET have the disadvantage of relatively short effective distances of quenching, limiting such probes to lengths of 15 to 25 bases or internal labelling with a dye is required.

The design, synthesis and use of the probes of the invention is simple and straightforward, resulting in simpler and more straightforward separation-free hybridisation assays and real-time PCR schemes based on the probes of the invention.

A variety of chemical structures, which do not quench conventional fluorescent dyes, appear to be efficient quenchers for the phosphorescent labels of the invention. For example, we have found that common fluorescent dyes such as FITC, Rhodamine Green, Cy5, as well as some small chemical moieties, such as dinitrophenyl, efficiently quench the phosphorescent porphyrin labels. Such quenching is dependent on the probe conformation and its change upon hybridisation. Common dark quenchers such as dabcyl, QSY™ family and other well known quenchers were also seen to work efficiently with these labels. We have shown that quenching of the phosphorescent porphyrins in solutions by these compounds proceeds quite efficiently (Stem-Volmer constants may reach 10⁶-10⁵ M⁻¹), and is greatly enhanced in the hybridisation and close proximity systems described above.

We have also found that nucleic acids themselves as well as individual bases have practically no quenching effect on the phosphorescent porphyrin labels of the invention. This is very advantageous for the application of the probes. This is not always the case for other long-decay luminescent labels. For example, oligos labelled with terbium(III)-chelate were reported to alter their signal upon hybridisation to unlabelled complementary sequences (Nurmi, J. et al.—A New label technology for the detection of specific polymerase chain reaction products in a closed tube. Nucleic Acid Res., 2000, v. 28, pE28).

Due to the minimal quenching by natural bases and by sample components, and a broad choice of quenchers including small-size chemical structures, strong quenching in tri-functional oligos in single-stranded conformation and minor quenching in double-stranded conformation, the user is provided with greater flexibility in the design of probes of the invention and corresponding separation-free hybridisation assays using these phosphorescent labels and probes. In particular, probes which have minimal interference on hybridisation, amplification and enzymatic elongation of nucleic acids and which produce sufficiently large and easily detectable signal change upon hybridisation to their targets can be designed and prepared in a simpler and more reliable fashion. Some nucleotide analogs and modified bases with quenching ability can also be incorporated within the probe sequence at a specific location with respect to the phosphorescent label.

In addition, we have found that in the double-labelled oligonucleotide probes the phosphorescent porphyrin labels are effectively self-quenched. This fact can be exploited in corresponding proximity quenching assay schemes. In particular, the use of only one dye simplifies the probe chemistry and enhances the signal from the probe by having two porphyrin labels, both working as the reporter and the quencher at the same time.

In previous studies (Vanderkooi, J. M., Maniara, G., Green, T. J., Wilson, D. F.—J. Biol. Chem. 1987, v.262, p. 5476-5482), it was found that self-quenching of phosphorescent metalloporphyrins in solutions is significant, but it vanishes at dye concentrations below 1 μM. However, when two metalloporphyrin labels are bound to a biopolymer such as single-stranded oligonucleotide, their self-quenching was shown to be greatly enhanced. This is due to the relatively high local concentration of the label in the vicinity of the probe (micro-volume), sufficient flexibility, intra-molecular dynamics and possible stacking interactions within the probe.

In the invention, we have shown that in such tri-functional probes self-quenching appears to be strong both at very low (picomolar) and high (micromolar) concentrations of the probe. This allows the tri-functional probes to be used for the detection of nucleic acids in solution in a very similar way as using the probes described above containing special quencher as a second label. Such probes are simpler than molecular beacons and TaqMan probes as they require incorporation of only one type of label at two specific sites (usually 5′- and 3′-termini) and do not require an additional “stem region”.

Furthermore, using the probes of the invention in hybridisation experiments in solution, one can determine, whether the target hybridising to the probe comprises a perfect complement or contains mismatches, such as single-point nucleotide polymorphism (SNP). Quite distinct hybridization patterns and temperature profiles of the phosphorescent signal are produced in such cases.

It was also found that the probes of the invention, which have characteristic features as described herein with the examples of oligonucleotide recognition structures, may also be designed on the basis of specific oligopeptide sequences. Such a probe, when recognised in solution by the corresponding enzyme or receptor, also alters the degree of quenching of the reporter label and, hence, the phosphorescent signal obtained from the probe. One possible mechanism of signal alteration is the probe chemical modification or cleavage by a target enzyme (e.g. a protease), which breaks the link between the reporter and the quencher, releasing two fragments of the probe eliminating proximity quenching effects. Another mechanism is binding of the probe to the target or probe chemical modification such as phosphorylation or dephosphorylation by a phosphatase or kinase enzyme, which affect the probe conformation and the degree of interaction between the reporter and the quencher moieties. In the absence of target the probe usually remains ‘dark’ in solution, while in the presence of target the probe gets cleaved, bound or modified and produces a highly phosphorescent form. In this case, the reporter and the quencher are usually located some distance apart from the cleavage or binding region in the probe. The corresponding signal change or pattern produced by the probe can be used for identification of the target and its quantification. This approach is particularly useful for the measurement of the activity and inhibition of important enzymes, such as proteases, kinases, phosphatases, esterases, and their inhibitors or activators.

The preferred probes of the invention are those in which at least one of the labels comprises a phosphorescent Pt(II)- or Pd(II)-complex of a porphyrin dye or a closely related structure such as chlorin, benzochlorin, porphyrin-ketone. Some other dyes, which have strong to moderate phosphorescence at room temperature in aqueous solutions and satisfy the hereinbefore described label requirements, may also be used as labels.

The preferred probes of the invention are those which can be produced by simple chemical procedures and which are easy to prepare in a pure, homogeneous and well characterised form. It is therefore advantageous for the phosphorescent label to be available as a monofunctional labelling reagent. This facilitates the preparation of the probe through chemical synthesis and purification. If labelling is carried out in aqueous solutions it is desirable for the label to be sufficiently hydrophilic and water-soluble and to have minimal tendency for non-specific binding to surfaces and sample components. Examples of such preferred labels include polycarboxylic metalloporphyrins such as Pt- and Pd-coproporphyrins (PtCP and PdCP), Pt- and Pd-tetrakis-(p-carboxyphenyl)porphin, derivatives or close analogs of these compounds. The most preferred phosphorescent labels and labelling reagents for making the probes of the invention are the monofunctional reactive derivatives of PtCP and PdCP, such as those described in U.S. Pat. No. 6,582,930. For example, monosubstituted 4-isothiocyanatophenyl-derivatives PtCP and PdCP may be easily conjugated with synthetic oligos bearing standard amino-modifications at 3′-end, 5′-end or within the sequences (O'Sullivan, et al. Nucleic Acid Res., 2002, v. 30, p.E1-7), to produce stable conjugates. Similarly, corresponding monofunctional maleimide derivatives of PtCP and PdCP may be conjugated with thiol-modified oligonucleotides. In a similar fashion, the second dye molecule or the quencher may be attached to the probe at the required site.

Alternatively, the phosphorescent reporter label and the effector/quencher may be incorporated in the probe sequence (at either end or internally) during the solid-phase oligonucleotide synthesis. This is usually carried out according to standard procedures, for example using a phosphoramidate method and corresponding phosphoramidate derivatives of mononucleotides and the labels.

One of the preferred types of probe of the invention comprises a specific oligonucleotide sequence with two labels attached to its 5′- and 3′-ends, with at least one of these labels being a phosphorescent label, such as Pt- or Pd-porphyrin. For some applications the tri-functional probes with the phosphorescent reporter dye and/or the effector/quencher incorporated internally may be used and are preferred.

Examples of efficient pairs of labels (phosphorescent reporter and effector) for the oligonucleotide probes of the invention are: 3′-PtCP and 5′-PtCP; 3′-PtCP and 5′-dinitrophenyl (DNP); 3′-PtCP and 5′-dabcyl; 3′-PtCP and 5′-QSY-7; 5′-PtCP and 3′-DNP; 5′-PtCP and 3′-dabcyl; 5′-PtCP and 3′-QSY-7; 3′-PdCP and 5′-PdCP; 3′-PdCP and 5′-DNP; 3′-PdCP and 5′-dabcyl; 3′-PdCP and 5′-QSY-7; 5′-PdCP and 3′-DNP; 5′-PdCP and 3′-dabcyl; 5′-PdCP and 3′-QSY-7. The preferred pairs of labels for these probes are: 3′-PtCP and 5′-PtCP; 3′-PdCP and 5′-PdCP.

The optimal length of the oligonucleotide probe is determined by a number of factors such as the target sequence, labels used, label attachment site, format and other practical requirements of a particular assay. It appears that 20-50-mer probes are the most effective and convenient for most applications and overall they produce better results. However, longer or shorter probes may also be used.

The method of detection of target nucleic acid sequences using hybridisation probes of the invention includes the following main steps:
preparation of sample containing target nucleic acid sequence for the analysis. This may include isolation, purification and enrichment of the initial biomaterial and preparation of fraction containing target;
addition to said sample of the probe of the invention specific to the target, under the conditions which favour the process of recognition of the target by the probe and hybridisation to it (buffer, temperature, additives, probe concentration, etc.);
Measurement of the probe phosphorescent signal from the sample and its changes associated with the target recognition process;
Quantification of the amount of target on the basis of these signal changes.

The method may be further modified by coupling it with a nucleic acid amplification process, for example polymerase chain reaction or other common schemes of nucleic acid amplification. Such processes and assay schemes, which are well known to specialists in this area, include for example the addition of two oligonucleotide primers (forward and reverse) specific to the particular part within target sequence, polymerase enzyme, its substrates (a mixture of nucleotide bases) in corresponding buffer system, additives, and incubation of the sample under certain temperature modes (cycles of annealing, elongation and melting) for a reasonable period of time. This method generally resembles the well-established formats of real-time PCR, for example ‘molecular beacons’, TaqMan. The phosphorescent probe of the invention generates changes of phosphorescent signal in response to the increasing amount of target produced in the amplification process. To achieve the detection and quantification of RNA targets, the process is usually coupled with reverse transcription which precedes the amplification.

The method of the invention may be further modified to achieve differentiation between the target which is fully complementary to the probe and the one which bears mismatch(es). The general design of such assays is well-known for specialists working in these areas.

Another type of probe of the invention comprises an oligonucleotide sequence specific to the target which contains a phosphorescent label attached to its 5′-end and a quencher incorporated internally into the probe. The probe not only alters its signal upon recognition of target nucleic acid sequence, but its remaining part serves as one of the primers in the amplification of the target sequence. For such probes the preferred quenchers are small-size labels which have minimal interference on the ability of such probe to act as a primer in the amplification process. In this case target amplification and detection require only one probe and one primer, so that the whole assay becomes simpler than classical real-time PCR schemes with short-lived fluorescent probes, which normally require two primers and a probe.

Yet another probe of the invention comprises an oligopeptide sequence, which has a similar design to the above oligonucleotide probes, i.e. contains in its structure a long-lived phosphorescent reporter label and the quencher moiety, and which also produces a distinct signal response upon binding to or cleavage by the corresponding protein such as an enzyme or receptor. The labels are usually attached to different parts of the oligopeptide backbone using the appropriate functional groups of the oligopeptide, such as primary amino group of lysine residues or N-termini, thiol group of cysteine residues, C-termini carboxy group, using corresponding conjugation chemistries.

Such peptide probes are useful for measurement of the activity and inhibition of corresponding enzyme(s), which also can be carried out in solution without the need of separating the free and bound/cleaved forms. The phosphorescent label is initially quenched by the quencher moiety located in close proximity to it. Upon the probe binding to the receptor target or upon its cleavage by the enzyme, the degree of interaction between the reporter label and the quencher is changing, due to spatial separation or increased probe stringency due to binding process. For example, if the probe is cleaved by an enzyme to produce two separate fragments, one with the reporter and the other with the quencher moiety, which are released in solution, this enhances the probe signal. This can be correlated with the amount of target present in the sample. Such a probe and method may be used to determine the activity of enzymes in test samples, their catalytic characteristics such as Vₘₐₓ and Kₘ, as well as the action of other compounds on the these enzymes causing their inhibition or activation. Preferably the pairs of labels that may be used as the reporter and the quencher in such probes include: PtCP and dabcyl; PtCP and QSY-7™; PdCP and dabcyl; PdCP and QSY-7™.

Furthermore, for the probes acting as phosphorogenic enzyme substrates it is advantageous to have an intrinsic rather than extrinsic effector/quencher within the oligopeptide sequence which alters the signal of the phosphorescent reporter label. We have found that among twenty natural amino-acid residues composing proteins and polypeptides, a few have the ability to quench the phosphorescent porphyrin label located in close proximity to them. Thus, phosphorescence of platinum(II)-coproporphyrin label in conjugates with histidine, lysine and tyrosine was shown to be considerably quenched, whereas the other natural amino acids had no significant quenching effect on the porphyrin label. These findings allow such chemical structures to be used as intrinsic quenchers in the probes of the invention. This approach results in an alternative and improved probe. Such oligopeptide probes are simple and easy to make.

The general design of the oligonucleotide and oligopeptide probes hereinbefore described and illustrated in the examples may be applied to other chemical or biological recognition structures. The examples of such structures and corresponding probes include those based on oligosaccharides, peptide nucleic acids (PNAs), other biopolymers and biologically active compounds.

Measurement of the signal of the probes of the invention in corresponding assays may be achieved by prompt or time-resolved fluorescence. Time-resolved fluorescence is the preferred detection method, as it provides greater sensitivity and selectivity of probe detection in complex biological samples, and it reduces interference by light scattering, sample autofluorescence or other fluorescent compounds present in the sample. It also allows more efficient multiplexing of probes and assays of the invention with other probes using time and wavelength discrimination. High sensitivity of the probes based on the phosphorescent porphyrin labels also allows miniaturization and reduction of sample volume in such assays.

Overall, the probes and methods of the invention overcome some of the limitations of the existing probes and assays, provide improved assay performance and allow the development of new assay formats. The invention provides simpler, more flexible and cheaper oligonucleotide and oligopeptide probes and corresponding separation-free hybridisation and enzymatic assays, which are not as dependent on various special requirements to the probe chemical composition, structural organisation, assay design and conditions.

Compared to similar probes based on short-decay fluorescent labels, these probes have clear advantages. In particular, they do not require special efforts to bring the label and the effector/quencher close together. Probes of the invention may be quite long (e.g. 80 nucleotide bases), while still retaining strong quenching by the internal quencher. This is difficult to achieve with conventional probes based on the FRET mechanism. In some of the probes only one extrinsic label is required, as the quencher can be either the same label (self-quenching) or an intrinsic quencher within the probe structure (internal quenching).

The probes and assays of the invention are easy to design and can provide high sensitivity and selectivity, particularly when using time-resolved fluorescent detection with time and wavelength discrimination. They can complement existing fluorescent probes used in separation-free bioassays and be coupled with them to allow assay multiplexing for simultaneous detection of several targets in one sample.

The invention provides a means for the detection of nucleic acids in solution using hybridisation probes comprising phosphorescent labels. The invention also provides for the design of phosphorescent probes and their use in separation-free hybridisation assays.

The invention further provides optimised pairs of chemical structures for use as the reporter and the quencher in hybridisation probes. Such probes produce optimal signal response upon recognition of their target. At the same time they have minimal interference on the hybridisation to the target and are easy to design, make and use.

More specifically, the invention provides a ‘linear’ dual-labelled probe, which contains a specific oligonucleotide sequence with a phosphorescent reporter label and effector attached to its termini (5′ and 3′), for use in separation-free hybridisation assays. One additional feature of die probe of the invention is its ability to serve as a primer in the amplification of target sequences which produces changes in its phosphorescent signal in the course of such amplification. To preserve their ability to prime the amplification of target nucleic acid by polymerase enzyme, such probes may have their 3′-end unmodified, while containing one of the labels internally.

The invention also describes a method of detection and quantification of target nucleic acid sequences in solution on the basis of changes of phosphorescent signal originating from such a probe upon the addition of sample containing target sequence, which is specifically recognised by the probe. Target recognition by the probe and hybridisation produce a luminescent signal or signal change, which can be correlated to the amount of target.

The invention also describes a method for the detection of mismatches and single-point mutations in the amplified nucleic acid sequences, using these phosphorescent probes and detection methods.

Furthermore, the invention describes a method of monitoring amplification of target nucleic acid sequences in real time PCR in a homogenous solution.

The invention also describes a method of multiplexing of separation-free hybridisation assays and a method of performing such assays, in which several hybridisation probes, each labelled with a different phosphorescent and/or reporter dye, are used simultaneously in one assay tube. Each specific luminescent signal is determined based on spectral and time discrimination of each individual label in a mixture.

The invention also provides a probe which produces signal change upon its cleavage (e.g. by an enzyme), which breaks the integrity of the probe and linkage between the reporter and quencher to one chemical species. Such probes may be used for monitoring the activity of important enzymes (used as substrates or substrate analogs), or the process of enzymatic elongation of a polynucleotide chain by certain polymerase enzymes (e.g. 5′-endonuclease activity of Taq polymerase and TaqMan® assays).

The invention has multiple applications and may be used for example in areas of molecular and cell biology, medicine, in vitro diagnostics, biotechnology, genetics, drug discovery, food and pharmaceutical.

The invention will be more clearly understood from the following examples

### EXAMPLE A

### Labelling of Oligonucleotides with Phosphorescent Metalloporphyrins

Synthetic oligonucleotides (purity tested by MALDI) containing the quencher and/or primary amino modifications (5′, 3′ or internal) were obtained from different suppliers (e.g. MWG-Biotech). A stock of quencher-labelled, amino-modified oligonucleotide was diluted in 0.1M borate buffer, pH 9.5 to a concentration of 0.18 mM. p-isothiocyanatophenyl derivative of platinum(II)-coproporphyrin I (PtCP-NCS) was dissolved in DMSO (18 mM) and then aliqouted into a clean, dry glass vial insert. The solution of oligonucleotide was then added to the vial to achieve a final concentration of 90 μM and dye/oligonucleotide molar ratio 14:1. The vial was then crimped to seal and incubated overnight at 37° C. in a hybridisation oven under continuous shaking. Chromatographic analysis and purification of reaction mixtures were carried out by reverse phase HPLC, using Agilent 1100 series system and Discovery™ C-18 column, 250 mm×4.6 mm. The peaks containing labelled oligonucleotides were identified by spectral analysis on the diode-array photometric detector, collected and further purified on a NAP5™ gel filtration column using 0.1M Tris buffer, pH 7.4 containing 0.3M NaCl. The principal fractions collected from this step were then desalted on a NAP5™ gel filtration column using water. Fractions with characteristic absorption of the conjugate were dried by vacuum centrifugation, re-suspended in 10 mM Tris buffer, pH 8.5 containing 50 mM KCl and 1.5 mM MgCl₂ to a concentration of 10 uM, aliquoted and stored frozen at −70° C.

Similarly, oligonucleotide probes containing the phosphorescent palladium(II)-coproporphyrin label were synthesized, using PdCP-NCS as labelling reagent. Alternatively, thiol-modified oligonucleotides were labelled with monofunctional maleimide derivatives of Pt- and Pd-coproporphyrins, using similar procedure and neutral buffer, pH 7.8.

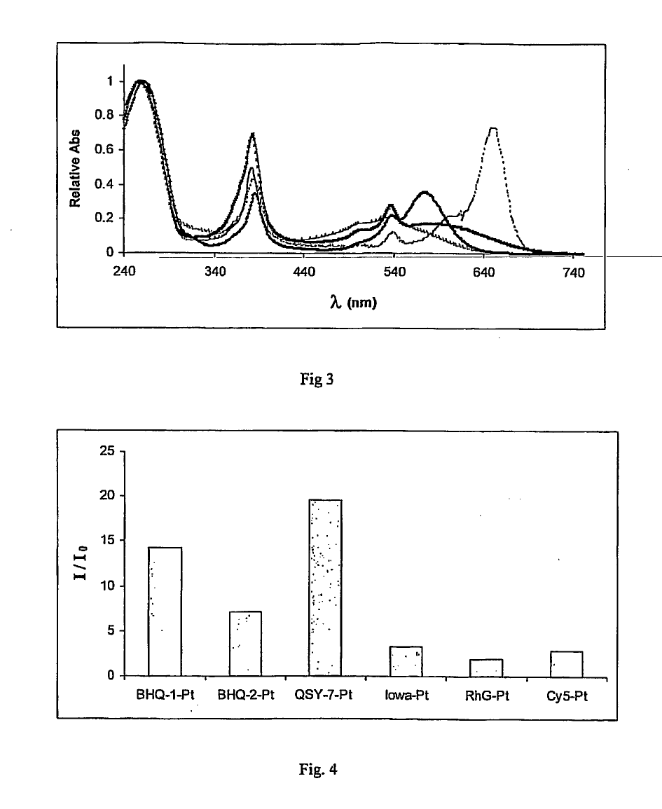

Absorption spectra of several dual-labelled probes after purification procedure are shown in FIG. 3. The all display characteristic absorption bands due to the oligo backbone (maximum at ˜260 nm), the PtCP label (peaks at ˜380 and 535 nm), and the quencher label.

Structures of some dual labelled probes containing PtCP reporter labels and different quencher moieties are given in Table 2 below.

### EXAMPLE B

### Quenching of the Phosphorescent Labels in Close Proximity Formats

Close proximity quenching was investigated using pairs of complementary oligonucleotides, one labelled with PtCP/PdCP and the other with the quencher. A series of hybridisation experiments in buffer solution were conducted to evaluate a range of potential quenchers. Hybridisation of two complementary terminal labelled oligonucleotides brings the two labels into close proximity, facilitating quenching of the PtCP signal. By varying the labelling site (5′ or 3′) and/or length of oligo(s), it is possible to vary the distance between the reporter label and the quencher in the resulting duplex structures, and examine its effect on the quenching.

The degree of quenching of phosphorescence of the 5′-PtCP labelled oligonucleotide upon the addition of a two-fold molar excess of complementary oligo labelled at 3′-end with the quencher (i.e. the label and the quencher are adjacent to each other in the duplex) was assessed using excitation of PtCP both at 381 nm (i.e. So→S2) and 535 nm (So→S1). Table 1 shows that a higher degree of quenching was observed for all the studied quenchers upon excitation of PtCP at the Soret band, when compared to excitation at 535 nm. This indicates that higher energy states of MeCP labels contribute to their quenching in such close proximity formats. Soret band excitation, which is frequently used for the detection of MeCP phosphorescence as it has higher molar absorptivity and produces higher levels of phosphorescence, produces higher degree of quenching.

Furthermore, changes in the phosphorescence intensity and lifetime of PtCP label upon interaction with the quencher are far from being synchronous, phosphorescence intensity is affected much greater. Also there is practically no correlation between spectral overlap integral of the reporter label and the quencher and the degree of quenching. Table 1 gives the proximity quenching of a PtCP label attached to oligonucleotide (model system). All this indicates complex mechanisms of quenching of the phosphorescent MeCP labels.

Similar results were obtained with oligonucleotides bearing PdCP label.

As previously described in the text and illustrated in FIG. 2, distance dependence of quenching of MeCP labels in such systems is also seen to be very characteristic and different from what is usually observed with conventional fluorescent labels.

TABLE 1

| | % Residual | % Residual | | |
|---|---|---|---|---|
| | Intensity, | Intensity, | % Residual | |
| | excitation at | excitation at | Lifetime, | % Overlap |
| Quencher | 381 nm | 535 nm | (τ), us | (648 nm) |
| Pd | 15.0 | 20.0 | 95.6 | 2.0 |
| CY5 | 27.0 | 39.6 | 64.4 | 90.3 |
| QSY-7 | 7.5 | 12.0 | 59.2 | 5.0 |
| RhG | 25.0 | 37.5 | 96.1 | 3.5 |
| Dabsyl | 27.5 | 34.0 | 89.1 | 1.3 |
| CuCP | 9.7 | 14.0 | 88.8 | 0.0 |
| Pacific Blue | 85.0 | 88.0 | NM | 0.0 |
| Unlabelled | 92.0 | 98.0 | 100.0 | NA |

### EXAMPLE 1

### Phosphorescent Properties of the Tri-Functional Oligonucleotide Probes

Structures of some representative dual-labelled (tri-functional) oligonucleotide probes and their phosphorescent properties/characteristics in the free form and in complex with complementary target (single-stranded double-stranded conformations, respectively) in solution are given below in Table 2.

TB 1 probe sequences of different length were selected from a specific sequence of the rpoB gene of Mycobacterium Tuberculosis. The region of interest (bases 11063-11367 of the rpoB gene) contains a high number of single base pair mismatches which confer rifampicin resistance on the bacterial strain. Sequences TB 2, TB 3 and TB 4 are base pair mismatches and alternative probe sequences selected form the rpoB gene sequence of interest at random.

In comparison with free PtCP and with single-labelled oligos (FIG. 2), the phosphorescence of dual-labelled oligos in aqueous solution in single-stranded conformation is quenched by 3-30 times. Maximal quenching is observed for the probes 20-25 bases long, quenching remains considerable (several-fold) for the probes 50 bases long and even longer. As opposed to the phosphorescence quantum yield (or intensity), lifetime of PtCP label is quenched much less.

The degree of quenching of the dual-labelled oligo probes is dependent on the quencher dye. FIG. 4 shows that QSY-7 and BHQ-1 appear to be among the best quenchers for PtCP label. For these probe structures and quenchers there is again no significant correlation between quenching efficiency and overlap integrals of their absorbance and PtCP emission. Within uM-nM range the degree of quenching is not dependent on die probe concentration.

TABLE 2

| | | | Enhancement | | | |
|---|---|---|---|---|---|---|
| Olgo name | Sequence | Φₛₛ | factor* | τₛₛ, us | τ_{ds}, us | τ_{ds}/τₛₛ |
| TB1-18mer- | 5′ PtCP - CAC GTC GCG GAC | 0.065 | 9.17 | 39 | 57 | 1.46 |
| QSY7-Pt | CTC CAG - QSY7 3′ | | | | | |
| TB1-21mer- | 5′ PtCP - GCA CGT CGC GGA | 0.065 | 13.09 | 50 | 72 | 1.44 |
| QSY7-Pt | CCT CCA GCC - QSY7 3′ | | | | | |
| TB1-23mer- | 5′ PtCP - TGC ACG TCG CGG | 0.032 | 32.65 | 37 | 63 | 1.70 |
| QSY7-Pt | ACC TCC AGC CC - QSY7 3′ | | | | | |
| TB1-25mer- | 5′ PtCP - TGC ACG TCG CGG | 0.058 | 21.59 | 38 | 59 | 1.55 |
| QSY7-Pt | ACC TCC AGC CCG G - QSY7 3′ | | | | | |
| TB1-30mer- | 5′ PtCP - GGG TGC ACG TCG | 0.149 | 3.21 | 29 | 43 | 1.48 |
| QSY7-Pt | CGG ACC TCC AGC CCG GCA - | | | | | |
| | QSY7 3′ | | | | | |
| TB1-50mer- | 5′ PtCP - TAG TGC GAC GGG | 0.131 | 3.52 | 45 | 68 | 1.51 |
| QSY7-Pt | TGC ACG TCG CGG ACC TCC | | | | | |
| | AGC CCG GCA CGC TCA CGT | | | | | |
| | GA - QSY7 3′ | | | | | |
| TB1-23mer- | 5′ PtCP - TGC ACG TCG CGG | 0.092 | 8.83 | 36.5 | 65 | 1.78 |
| IowaBlack- | ACC TCC AGC CC - Iowa 3′ | | | | | |
| Pt | | | | | | |
| TB2-23mer- | 5′ Alexa - TGC ACG TCG CGG | 1.10 | 1.19 | 44.5 | 59 | 1.32 |
| Alexa 647-Pt | ACC TCC AGC CC - PtCP 3′ | | | | | |
| TB3-23mer- | 5′ PtCP - TGC ACG TCG CGG | 0.98 | 1.17 | 52.5 | 58 | 1.10 |
| RhG-Pt | ACC TCC AGC CC - RhG 3′ | | | | | |
| TB4-23mer- | 5′ PtCP - TTG ACC CAC AAG | 0.11 | 6.29 | 50 | 63 | 1.26 |
| BHQ1-Pt | CGC CGA CTG TC - BHQ1 3′ | | | | | |
| TB4-23mer- | 5′ PtCP - TTG ACC CAC AAG | 0.08 | 7.54 | 50 | 61 | 1.22 |
| BHQ2-Pt | CGC CGA CTG TC - BHQ2 3′ | | | | | |

*increase of the phosphorescence intensity upon probe hybridization to complementary target.
Φₛₛ - relative phosphorescence quantum yields of dual-labelled oligonucleotide probes, with respect to the single-labelled oligonucleotide with PtCP label, both free in solution in single-stranded conformation;
τₛₛ, τ_{ds}—phosphorescence lifetimes of oligos in the single-stranded and double-stranded conformations, respectively;
Conditions: 10 mM tris buffer, pH 7.8 containing 50 mM KCl, 1.5 mM MhCl2, 100 mM Na2SO3, 23° C.

These results indicate that for such dual-labelled single-stranded oligonucleotide structures dissolved in aqueous solution static or pseudo-static quenching plays a major role, whereas classical dynamic quenching or resonance energy transfer are less significant.

### EXAMPLE 2

### Hybridization of the Phosphorescent Tri-Functional Oligonucleotide Probes with Complementary Targets in Solution: Single-Stranded vs Double-Stranded Conformation

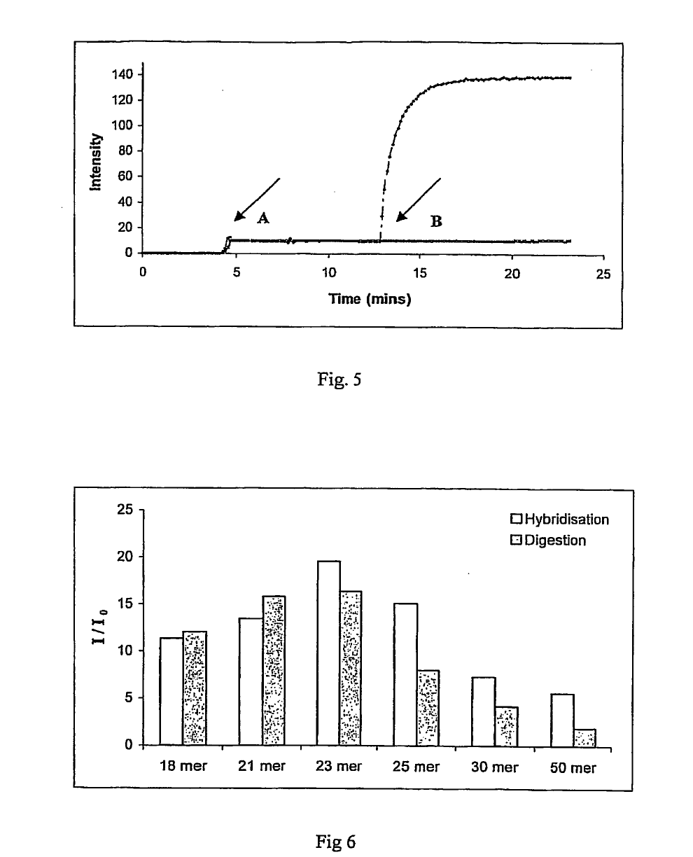

Upon the addition of complementary target sequence to a solution of the tri-functional phosphorescent oligo probe, large (many-fold) enhancement of the phosphorescence was observed for all the probes, as shown in FIG. 5.

This indicates that the probe phosphorescence is considerably quenched only in the single-stranded conformation. In the double-stranded conformation the probe quenching is very minor, if any. Probe phosphorescence in the double-stranded conformation appears to be close to that of the free PtCP label or single-labelled oligo in solution.

The dependence between the degree of signal enhancement and probe length has a bell shape, as shown in FIG. 6. Such pattern is very characteristic and it differs considerably from the other types of hybridization probes, such a TaqMan and ‘molecular beacons’.

FIG. 6 also shows that recognition of the single-stranded tri-functional probe by nuclease enzyme resulting in the probe digestion also restores the phosphorescence of the PtCP label due to the elimination of its proximity quenching by the quencher. In this case, signal increase produced by the probe is related to its cleavage.

Temperature dependence of quenching of the tri-functional phosphorescent oligonucleotide probes (TB1-QSY-7-PtCP probes of different length) are shown in Table 3 below. One can see that the enhancement of phosphorescence upon hybridization with target sequence remain strong at elevated temperatures, up to the point when the probe melting temperature is reached. These results show that quenching of the probe is dependent on its conformation and change in conformation upon recognition of the target produces a distinct phosphorescent response.

TABLE 3

| Temp | | | | | | |
|---|---|---|---|---|---|---|
| (° C.) | 18mer | 21mer | 25mer | 23mer | 30mer | 50mer |
| 20 | 10.80 | 11.36 | 19.20 | 20.16 | 7.70 | 7.75 |
| 30 | 9.01 | 8.94 | 18.29 | 29.14 | 7.90 | 7.90 |
| 40 | 11.74 | 14.44 | 16.48 | 17.49 | 7.09 | 7.04 |
| 50 | 8.66 | 12.48 | 16.98 | 16.12 | 7.11 | 7.06 |
| 60 | 8.25 | 12.47 | 14.50 | 26.95 | 7.20 | 6.34 |
| 70 | 3.42 | 10.16 | 11.58 | 21.48 | 7.68 | 6.28 |
| 80 | 1.20 | 1.52 | 7.51 | 4.61 | 4.73 | 3.30 |

Strong phosphorescence of the probe in the presence of target and strong quenching in the absence of target and large and specific signal change upon recognition of its target, such as complementary DNA sequence or nuclease enzyme, the probes of the invention can be used for the detection of specific DNA sequences in solution using homogeneous assay formats.

### EXAMPLE 3

### Application of the Tri-Functional Oligonucleotide Probes to the Detection of Single-Point Mismatches in the Target Sequence

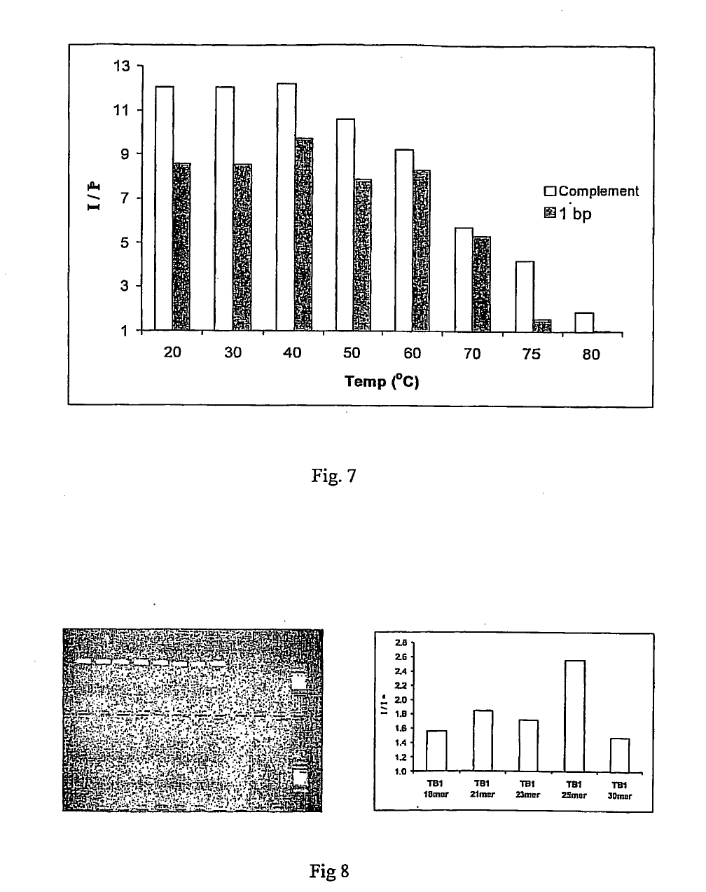

FIG. 7 shows the enhancement of the phosphorescent signal upon hybridization of the tri-functional oligonucleotide probe to its target at several different temperatures, and the effect of single base mismatch in target sequence.

### EXAMPLE 4

### Coupling of Target Amplification in a PCR with its Recognition by the Tri-Functional Phosphorescent Oligonucleotide Probe—Real-Time PCR Format

PCR was carried out on an Eppendorf Mastercycler® PCR block with heated lid using either HotMaster (Eppendorf) or individual reaction components (Bioline), in a final volume of 50 ul. Concentrations of primers were maintained at 0.2 uM and between 1 and 10 ng of template DNA was added to the reaction mixture. Amplification thermocycling was optimised for each individual system. TB1 probes set up as follows: 19-mer forward and reverse primers were designed to flank a 173 base pair region of genomic template DNA 94° C. for 2 min initial melting time followed by 35-40 cycles of; 58° C. for 1 min, 72° C. for 1 min and 94° C. for 0.5 min. A final 2-5 min step at 72° C. completed amplification. Negative controls containing all PCR reagents except template DNA were run simultaneously.

Samples of PCR reaction mixtures were run on a 1.5% agarose gel (in TAE) (50 mls approx) stained with ethidium bromide or SYBR® Gold (0.001% v/v) with 6× loading Dye (Promega) and electrophoresed for approximately 30 mins at 50 V on Fast Mini Horizontal Gel Unit (SciePlas). A 100 bp DNA ladder (Promega) was used as a molecular weight marker. DNA samples were visualized under UV illumination using a GelDoc™ system with accompanying GeneSnap™ software (Syngene). Measurement of probe incorporated PCR reaction mixtures was performed on a Victor® 2 multi-label counter using 40 ul of neat reaction mixture on a 384 well black plate.

As shown in FIG. 8, amplification of product was not significantly affected by the presence of the probe in the PCR reaction mixture. (HotMaster hot-start system, Eppendorf), UV-visualisation of PCR product by SYBR® Gold DNA staining indicated successful amplification of specific PCR product in the presence of all probes. Neat reaction mixtures were transferred to a 384 well black plate and measured on the Victor® 2 multi-label counter. Reaction mixtures were measured in the presence and absence of oxygen, using sodium sulfite as a chemical de-oxygenator. Although signal increased in the presence of sulfite, overall signal to noise ratios were not affected positively. End-point measurement of samples and comparison of positive and negative controls reveal a distinct and reproducible change of 2-3-fold increase in PtCP signal after PCR amplification. Signal changes are not of the same magnitude as in the model systems, which may be explained by the fact that the target was in double-stranded conformation. The results are comparable with existing systems, including those using long lived fluorescent lanthanide chelates.

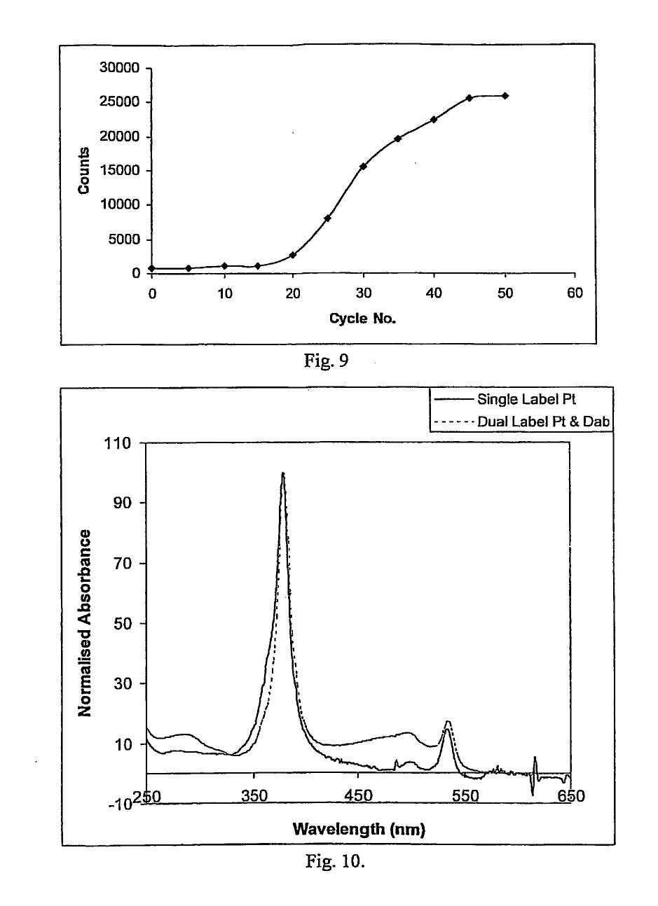

In another similar experiment, the probe was incorporated in a sample containing 1 ng of template DNA. The sample underwent PCR amplification with two primers specific to the sequence of IGF2 gene. During the PCR, small aliquots of sample were taken after every 5 cycles and analysed by time-resolved phosphorescence measurements on a plate reader. The profile of the probe phosphorescent signal is shown in FIG. 9. One can see a considerable signal increase over time (cycle No), which reflects the increased amounts of target DNA amplified in the PCR.

### EXAMPLE 5

### Oligonucleotide Probe Based on the Two PtCP Labels and Self-Quenching

The 23-mer oligonucleotide probe (TB-1 sequence) bearing two amino modifications at 5′- and 3′-termini was dual-labelled with PtCP-NCS reagent using a two-step labelling protocol. The first labelling step carried out as described in Example 1 produced predominantly a single-labelled product, which was purified by HPLC, collected, pooled and dried on a vacuum centrifuge. This product was re-dissolved in carbonate buffer and labelling and purification procedure was repeated under the same conditions. Thus, dual-labelled oligonucleotide probe was produced (composition was confirmed by UV-VIS analysing the ratio of bands at 260 nm and 380 nm). Similarly to the probes described in example 3, this probe was also found to be quenched in its single-stranded conformation (self-quenching of two PtCP labels). Upon hybridisation to the complementary target in solution or upon cleavage by nuclease enzymes, the probe produced a considerable enhancement of its phosphorescent signal.

### EXAMPLE 6

### Synthesis of a Tri-Functional, Phosphorogenic Oligopeptide Substrate for Caspase-3 and Homogeneous Detection in Induced Cell Lines

The octameric peptide Ac-CDEVDAPIC-NH₂, containing the DEVD recognition motif for caspase-3, was purchased from Peptron (Korea). To limit non-specific reactions during labelling and cleavage the peptide was purchased with N-terminal acetyl and C-terminal amide modifications. The P1 lysine and P8 cysteine were chosen as functional targets for fluorophor and quencher labelling.

Labelling was carried out as a two-step process with primary labelling with the quencher moiety and secondary labelling with monofunctional malemide derivative of PtCP. The moiety chosen for optimal quenching of PtCP was 4-[4 (dimethylamino)phenylazo]benzoic acid N-succinimidyl ester (Dabcyl, Fluka).

Labelling via the P1 lysine residue was achieved using a five molar excess of peptide in 0.1M sodium borate, pH 8.4. The reaction mixture was incubated for one hour shaking at room temperature followed by isolation and identification of the dabcyl-labelled product by chromatographic separation on an Agilent 1100 HPLC working in reversed phase using Discovery™ C-18 (5 μm, 250 mm×4.6 mm) column. The dabcyl-labelled peptide was eluted using a gradient of 0-100% acetonitrile in 0.1M triethylammonium acetate CIEAA), pH 6.5, in 21 min, with the product identified by its absorbance maximum at 453 nm giving a peak at 10.37 min. Secondary labelling of the peptide with PtCP-malemide was carried out in 0.1M sodium phosphate, pH 7.2 containing 0.15M sodium chloride using a ten molar excess of the porphyrin with respect to the 60 μM stock concentration of the primary labelled peptide in a 200 μl reaction volume, with incubation for four hours shaking at room temperature. Separation was carried out as above using a 0-70% gradient of acetonitrile in TEAA with dual wavelength monitoring at 380 nm (PtCP maximum) and 453 nm. The tri-functional peptide was identified as a peak at 12.163 min. The substrate was isolated, dried on a vacuum centrifuge and resuspended in assay buffer (50 mM HEPES, pH 7.2, containing 100 mM sodium chloride, 1 mM EDTA, 20% (v/v) glycerol and 0.1% (w/v) CHAPS). The absorbance spectrum of the tri-functional substrate is shown in FIG. 10.

The relative quantum yield of the tri-functional substrate (in above assay buffer) with respect to the bi-functional peptide, was calculated by measurement of time-resolved phosphorescence on both Victor² (Perldn Elmer) and ArcDia (Arctic Diagnostics) fluorometers, followed by normalising for concentration. On both instruments a delay time of 50 μs and a gate time of 100 us was used. Relative quantum yield values were estimated as 32% and 3.5% for both measurements respectively.

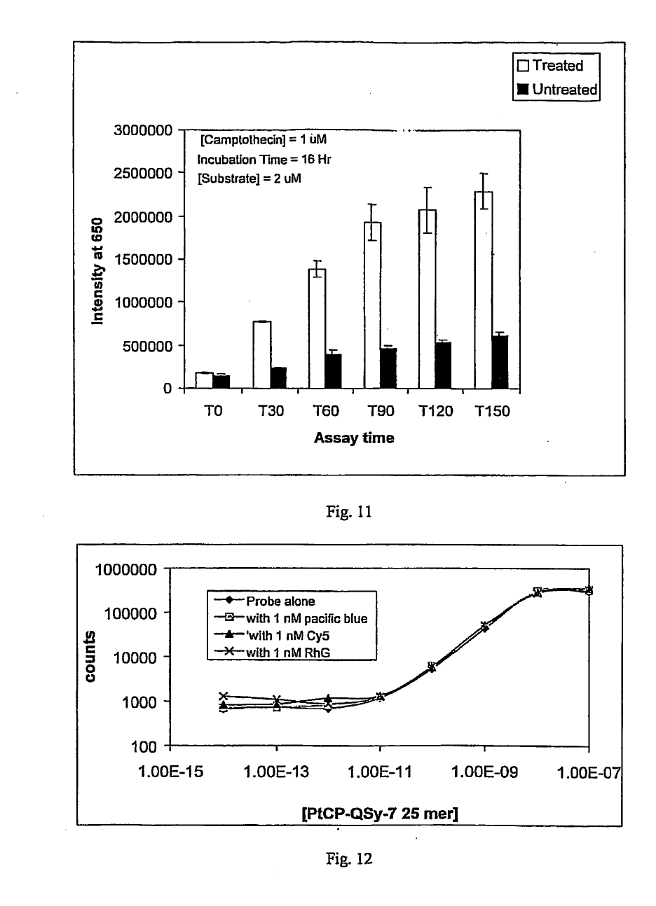

To test the phosphorogenic substrate as a potential homogeneous tracer for caspase-3 activity in induced cell lines, Jurkat T-cells were cultured in RPMI 1640 medium, containing 2 mM L-glutamine, 10% foetal bovine serum, 100 units/ml potassium penicillin and 100 μg/ml streptomycin sulfate, to a concentration greater the 1×10⁶ cells per ml. To induce apoptosis the cells were treated with 1 μM of the pro-apoptotic drug camptothecin followed by incubation at 37° C. for 16 hr. Both treated and untreated (control) cells were isolated by centrifugation at 1000 g for 5 min and resuspended in 200 μl assay buffer containing 20 mM β-mercaptoethanol and the non-specific protease inhibitors AEBSF (0.2 mM), leupeptin (10 mM) and pepstatin A (1 μM). Cell lysis, on the action of the CHAPS detergent, was carried out on ice with intermittent vortexing over a 30 min period. Cell lysate was isolated by centrifugation at 14 000 g for 10 min. The cleavage reaction was carried out by mixing an equal volume of 4 μM substrate and lysate, followed by incubation at 37° C. At specific time points, 10 μl aliquots were taken from the reaction vial and added to 990 μl of assay buffer, with 100 μl aliquots added to a black 96-well micro-titre plate. Dissolved oxygen was removed by addition of 10 μl of a glucose/glucose oxidase solution and measurement of phosphorescent signal was carried out on the Victor² as above. Results are shown in FIG. 11.

An approximate four fold increase in intensity is observed after 90 min with the treated sample (w.r.t. untreated) where apoptosis has been induced and thus caspase-3 is present. In this case, the enzyme cleaves at the C-terminal side of the P4 aspartate (D) residue thus liberating the PtCP from the proximity quenching effect of the dabcyl with resulting increase in intensity.

### EXAMPLE 7

### Selective Detection of the Phosphorescent Hybridisation Probes in the Presence of Other Fluorescent Probes

FIG. 12 shows that the presence of other fluorescent probes (oligos labelled with pacific blue, Rhodamine Green and Cy5 dyes) has no interference on the time-resolve fluorescence detection of the tri-functional phosphorescent probes of the invention. The probe comprises a 25-mer oligonucleotide labelled with PtCP at 5′-end and QSY-7 at 3′-end. Due to very efficient time and wavelength discrimination of the probes of the invention, they can be multiplexed with other fluorescent probes.

### EXAMPLE 8

### Oligopeptide Probes with Internal Amino Acid Quenchers

PtCP-NCS and PdCP-NCS were conjugated to each of the twenty natural amino acids. The phosphorescent labelling reagent was dissolved in 0.1 M carbonate buffer, pH 9.5, mixed with corresponding amino acid (10 mM final concentrations for both) and incubated for 4 h at 37° C. The conjugate was then purified by HPLC on a reverse phase column, dried on a vacuum centrifuge, re-dissolved in PBS and quantified spectrophotometrically. Phosphorescent properties of the resulting conjugates (quantum yields and lifetimes) were examined. For the conjugates with lysine, histidine and tyrosine, a considerable (40-90%) internal quenching phosphorescence intensity of the MeCP label was observed in aqueous buffers (e.g. PBS), with only minor quenching of lifetime. For all the other amino acid conjugates no significant quenching was seen.

Based on this information, several oligopeptide conjugates bearing PtCP label at one of the termini were designed and produced, using PtCP-NCS (reactive with primary amino groups of lysine residues) and PtCP-maleimide (reactive with HS-group of cysteine residues) as labelling reagents. Their structure and phosphorescent properties are summarised in the Table 4 and compared to those of free labels.

One can see that labelling with PtCP-NCS produce oligopeptide conjugates (compound 3) in which the PtCP label is quenched by the adjacent group (lysine). This is not very desirable as enzymatic cleavage site is usually located some distance away from the label. However, labelling with PtCP-MI via cysteine residue allowed us to avoid such internal quenching at the labelling site and produce bright conjugates (compound 4). Incorporation in oligopeptide sequence of amino acid residues, which were previously identified as quenchers of MeCP phosphorescence (e.g. tyrosine and histidine), produces conjugates with considerable quenching (compound 5, quenching amino acids are outlined in bold). Such conjugates, which bear intrinsic quencher(s) at some distance away from the label, have the ability to modulate their signal (enhancement of PtCP phosphorescence) upon cleavage. If required, such probes can be further labelled with extrinsic quencher such as dabcyl, which enhances the quenching effect (compound 6).

TABLE 4

| No | Compound | Relative* φ, % | τ, μs |
|---|---|---|---|
| 1. | PtCP-NCS | 98.6 | 91 |
| 2. | PtCP-MI | 10.58 | 89 |
| 3. | CDEVDAPK-PtCP (NCS) | 43.9 | 92 |
| 4. | PtCP-CDEVDAPK (MI) | 114.70 | 97 |
| 5. | PtCP-CEVYGMMHK (MI) | 17 | n/m |
| 6. | PtCP-CEVYGMMHK-dabcyl | 3.5 | n/m |

*Relative to PtCP in PBS;
n/m—not measured.

The invention is not limited to the embodiments hereinbefore described which may be varied in detail.

1-52. (canceled)

53. A probe comprising a supramolecular structure having:
a chemical or biological recognition moiety;
a phosphorescent reporter label; and
an effector moiety,
in which probe the label interacts with the effector so that the probe alters its phosphorescent characteristics upon recognition of a target.

54. The probe as claimed in claim 53 wherein the phosphorescent reporter label has an emission lifetime in the order of 1 μs to 10 ms.

55. The probe as claimed in claim 53 wherein the phosphorescent reporter label has an emission lifetime in the order of 10 μs to 1000 μs.

56. The probe as claimed in claim 53 wherein the phosphorescent reporter label is selected from a group of phosphorescent tetrapyrrolic compounds and their metallocomplexes.

57. The probe as claimed in claim 56 wherein the phosphorescent reporter label is selected from any one or more of phosphorescent metallocomplexes of porphyrins, chlorins, porphyrin-ketones and related structures.

58. The probe as claimed in claim 57 wherein the phosphorescent label is platinum(II)-porphyrin.

59. The probe as claimed in claim 57 wherein the phosphorescent label is platinum(II)-coproporphyrin.

60. The probe as claimed in claim 57 wherein the phosphorescent label is palladium(II)-porphyrin.

61. The probe as claimed in claim 57 wherein the phosphorescent label is palladium(II)-coproporphyrin.

62. The probe as claimed in claim 53 wherein the phosphorescent label is in the form of a monofunctional labelling reagent.

63. The probe as claimed in claim 53 wherein the effector moiety is selected from any one or more of dabcyl, QSY-7™, ‘black hole quenches’™, rhodamine green, FITC, Cy5™, and analogs thereof.

64. The probe as claimed in claim 53 wherein the effector moiety comprises a small-size chemical structure.

65. The probe as claimed in claim 64 wherein the effector moiety comprises a chemical structure less than 300 Daltons in size.

66. The probe as claimed in claim 64 wherein the effector moiety is selected from any one or more of dinitrophenol, a nitrophenol moiety and derivatives thereof.

67. The probe as claimed in claim 53 wherein the effector moiety is a modified nucleotide base.

68. The probe as claimed in claim 53 wherein the phosphorescent reporter label and the effector are both provided by the same chemical structure.

69. The probe as claimed in claim 68 wherein the reporter label and the effector both comprise a phosphorescent metalloporphyrin label.

70. The probe as claimed in claim 53 wherein the recognition moiety is a common biomolecular structure or a biopolymer.

71. The probe as claimed in claim 53 further comprising a spacer(s) linking the recognition moiety, the reporter label and the effector.

72. The probe as claimed in claim 71 wherein the spacer(s) is 2 to 18 atoms in length.

73. The probe as claimed in claim 53 wherein the reporter label is attached to one of the termini of a biopolymer acting as recognition moiety.

74. The probe as claimed in claim 53 wherein the recognition moiety comprises a biopolymer with the reporter label attached to one of its termini and the effector attached to the other termini.

75. The probe as claimed in claim 53 wherein the recognition moiety comprises a biopolymer with the reporter label attached to one of its termini and the effector attached internally.

76. The probe as claimed in claim 53 wherein the recognition moiety comprises a biopolymer with the effector attached to one of its termini and the reporter label attached internally.

77. The probe as claimed in claim 53 wherein the probe is quenched in its free form in solution.

78. The probe as claimed in claim 53 wherein the chemical or biological recognition moiety comprises a single-stranded oligonucleotide sequence.

79. The probe as claimed in claim 78 wherein the probe produces a phosphorescent signal response upon recognition of a complementary target, hybridisation and formation of a double-stranded structure with the target.

80. The probe as claimed in claim 78 wherein the reporter label and the effector are attached to the 5′- and 3′-ends respectively of the specific nucleic acid sequence.

81. The probe as claimed in claim 78 wherein the reporter label is attached to the 5′-end of the probe and the effector is incorporated internally or attached to one of the bases inside the probe sequence.

82. The probe as claimed in claim 78 wherein the probe is 15 to 100 bases long.

83. The probe as claimed in claim 82 wherein the probe is 20 to 50 bases long.

84. The probe as claimed in claim 78 wherein the probe has the ability to hybridise to a target and act as a primer in the process of elongation of the polynucleotide chain by a polymerase enzyme with the target acting as a template.

85. The probe as claimed in claim 78 wherein the reporter label is Pt-porphyrin and the internal effector is a modified nucleotide base.

86. The probe as claimed in claim 53 wherein the chemical or biological recognition moiety comprises an oligopeptide sequence.

87. The probe as claimed in claim 84 wherein quenching of the reporter label is affected by probe cleavage associated with the recognition process.

88. The probe as claimed in claim 87 wherein the probe is cleaved or modified by a specific enzyme.

89. The probe as claimed in claim 53 wherein the chemical or biological recognition moiety comprises a structure acting as an intrinsic quencher for the reporter label.

90. The phosphorescent probe as claimed in claim 89 wherein the intrinsic quencher for the phosphorescent metalloporphyrin label is a histidine residue within an oligopeptide sequence.

91. The phosphorescent probe as claimed in claim 90 wherein the intrinsic quencher for the phosphorescent porphyrin label is a tyrosine residue within an oligopeptide sequence.

92. The probe as claimed in claim 53 wherein the chemical or biological recognition moiety comprises a polysaccharide or a peptide nucleic acid.

93. A method for the detection of a chemical or biological species comprising the steps of:
providing a probe as claimed in any preceding claim;
exposing the probe to a sample containing a target species;
measuring the phosphorescent response of the probe on recognition of the target; and
qualifying and quantifying the target based on the measured phosphorescent signal.

94. The method as claimed in claim 93 comprising preparing a solution comprising the probe and mixing the probe solution with a sample solution containing a target.

95. The method as claimed in claim 93 comprising the process of amplifying the target.

96. The method as claimed in claim 93 wherein the target comprises a nucleotide sequence.

97. The method as claimed in claim 93 wherein the method comprises the recognition of a target sequence by the probe, amplification using a set of primers specific to a particular region within the target sequence and a polymerase chain reaction.

98. The method as claimed in claim 93 wherein the probe also acts as a primer.

99. The method as claimed in claim 93 wherein the probe is used to distinguish between complementary and non-complementary target nucleotide sequences.

100. The method as claimed in claim 93 wherein the probe is used to distinguish between a perfect complement and a single-point mismatch or polymorphism.

101. The method as claimed in claim 93 wherein target amplification and detection are carried out in a closed tube format.

102. The probe as claimed in claim 53 wherein the reporter label has two distinct excitation bands.

103. The method as claimed in claim 93 wherein the probe signal is measured by time resolved fluorescence.

104. The method as claimed in claim 103 wherein the probe is multiplexed with at least one other photoluminescent based probe.

105. The assay utilising a probe as claimed in claim 53 wherein the assay is selected from hybridisation, binding and enzymatic assays.

106. The assay as claimed in claim 105 wherein the assay is based on the use of close proximity quenching of a long-decay phosphorescent label.
